(19) United States
(12) Patent Application Publication
(10) Pub. No.: US 2025/0127384 A1
(43) Pub. Date: Apr. 24, 2025

(54) ENDOSCOPE COMPRISING A CLOT DISSECTING CHANNEL

(71) Applicant: AMBU A/S, Ballerup (DK)

(72) Inventor: Tim CLOSS, Augsburg (DE)

(73) Assignee: AMBU A/S, Ballerup (DK)

(21) Appl. No.: 18/924,586

(22) Filed: Oct. 23, 2024

(30) Foreign Application Priority Data
Oct. 24, 2023 (DE) 10 2023 129 148.3

(51) Int. Cl. A61B 1/015; A61B 1/00; A61B 1/018

(52) U.S. Cl. CPC A61B 1/015; A61B 1/00068; A61B 1/018

(57) ABSTRACT

An endoscope including: a handle; an insertion cord extending from the handle and including at least a distal tip; a working channel which during suction is configured to let a fluid and a mucus and a clot suctioned from the patient's body cavity flow therethrough; and a clot dissecting channel which is configured to eject a fluid stream for dissecting the clot before or while the clot enters the working channel, the clot dissecting channel being directed towards and opening up towards the working channel or a distal extension of the working channel in the distal tip.

### CROSS-REFERENCE TO RELATED APPLICATIONS

This application claims priority from and the benefit of German Patent Application No. 10 2023 129 148.3, filed Oct. 24, 2023; the disclosure of said application is incorporated by reference herein in its entirety.

### TECHNICAL FIELD

The present disclosure relates to endoscopes and, in particular, to an endoscope with a working channel.

### BACKGROUND

Endoscopes, both reusable and single-use, are typically used for visual examination and diagnosis of hollow organs and body cavities. Endoscopes include specialized instruments such as bronchoscopes, arthroscopes, colonoscopes, laparoscopes, gastroscopes, and duodenoscopes. An endoscope may comprise a working channel to sample fluids or tissue from the patient's body cavity or to guide a surgical instrument through it to a surgical location in the patient's body cavity.

When examining an object, such as a body cavity or hollow organ, with an endoscope it is desirable to have a clear and good view or visibility of the examined object. Visibility is often affected by presence of undesirable fluid, mucus or blood clots. It may be desirable to remove the undesirable matter, e.g. fluid, mucus or blood clots, to provide better visibility. It is thus desirable to remove such undesirable fluid, mucus or blood clots, as well as such tissue parts or particles, using a suction device, such as a vacuum pump.

Due to the limited size of the endoscope, in particular due to the limited diameter of the insertion cord of the endoscope, the working channel diameter has a small cross-section. The limited cross-section restricts the size of the blood clots that can be suctioned. Therefore, when a blood clot larger in size than the cross-section of the working channel is to be suctioned, the working channel may clog. Such a blockage needs to be resolved by the hospital staff before continuing the procedure, which is usually a time-consuming task. An operator of the endoscope can try to clear the blockage by introducing air or water rapidly with a syringe via the working channel to push the blockage towards the working channel exit. Alternatively, the operator can introduce a brush into the working channel to remove the blockage mechanically via the brush. A further alternative is to withdraw the endoscope out of the patient's body cavity to clear the blockage and to afterwards reintroduce the endoscope into the patient's body cavity. In emergency situations, the procedure time is critical and the known measures the operator can perform are not favorable to a desirable patient outcome.

### SUMMARY

The object of the present disclosure is to improve patient outcomes by reliably preventing blockage or unblocking the suction path of the endoscope, in particular when a clot or debris having a size larger than the cross-section of the working channel is suctioned into the working channel of the endoscope.

A first aspect of the disclosure relates to an endoscope. In one embodiment according to the first aspect, the endoscope comprises a clot dissecting channel directed to and opening towards a distal end of the working channel. The clot dissecting channel is configured to eject a fluid stream into the working channel to dissect obstructions (objects or particles), such as clots, before or while the obstructions enter the working channel.

Advantages of the clot dissecting channel include the deconstructing/dissection of obstructions to enable suctioning of a larger variety of particles, reduce the probability of the working channel getting blocked, clean instruments with the fluid flowing through the clot dissecting channel before retracting the instruments from the working channel, discharging a lubricant or other fluids through the distal tip exit of the working channel for purposes other than dissecting a clot, lubricating an instrument before retracting the instrument from the working channel to ease said withdrawal, and cleaning a biopsy sample, potentially to increase visibility and confirm that the correct tissue was removed. As used here, instruments include tools. As is evident from the potential advantages of the clot dissecting channel, the fluid discharged therethrough can be used for reasons other than to split obstructions. Thus, the fluid may be water or may comprise water and air or water and additives.

The endoscope according to the present embodiment may comprise a handle or interface, and an insertion cord extending from the handle or interface and configured to be inserted into the patient's body cavity. The insertion cord comprises an insertion tube, a bending section, a distal tip, and the working channel. The distal tip may comprise a camera and a light emitter. Example light emitters include light emitting diodes (LEDs) and optical fibers.

The endoscope comprises suction functionality which may comprise a suction valve at the handle fluidly coupled to the working channel. A vacuum source is connected to the working channel and vacuum through the working channel can be applied or switched off with the suction valve. The vacuum source may comprise a vacuum pump.

The handle may be substituted by the interface. Alternatively, the interface may be formed on the handle. Hence, in the context of this disclosure, the term handle may be used for a handle held by a hand of a user, such as a surgeon, or for an interface. The handle may contain control elements, such as a lever for controlling bending of the endoscope and ports/connectors for accessories such as a video processing apparatus or the vacuum source. The handle is preferably designed in such a way that it can be ergonomically gripped and operated by the user or operator or surgeon with just one hand. This means that the control elements on the handle are preferably arranged in such a way that the user can operate them with one hand without having to change a position of his or her hand. The interface may be prepared or provided to be connectable to a robotic arm.

The working channel is configured to transport fluid, mucus and clots, such as tissue parts or particles or debris, from the patient's body cavity. Furthermore, the working channel may be configured to guide a surgical instrument through it to a surgical location in the patient's body. For this purpose, the working channel may have a Y-connector in the handle that allows insertion of the surgical tool or instrument into the working channel.

The clot dissecting channel is a channel or passage or conduit in the distal tip. This clot dissecting channel is configured to discharge a clot dissecting fluid stream, which is a pointed and powerful fluid stream or water jet, towards an object blocking the working channel. The object may be a clot. The clot dissecting fluid stream is configured to dissect clots before or while they enter the working channel. Therefore, clots, particularly clots too large to pass through the working channel, are dissected into smaller clots that can be suctioned through the working channel.

According to the disclosure, an extension direction of the clot dissecting channel and an opening of the clot dissecting channel, through which the water jet or stream for dissecting the clots is ejected, are configured such that the water jet or stream is directed towards the working channel or a distal extension of the working channel. The distal extension of the working channel may be understood as a virtual elongation of the working channel along its axial direction. In other words, the distal extension of the working channel may be described as a virtual pipe-like structure, which is obtained, when the working channel is projected axially in the distal direction. Thus, the clot dissecting channel is directed to cross the suction path defined by the working channel inside the working channel, at the entrance of the working channel, i.e. at the distal opening of the working channel, or at a space slightly distal from the working channel entrance.

According to a preferred embodiment, the clot dissecting channel comprises or is an oblique channel inside the distal tip, the oblique channel guiding the fluid stream from a supply channel towards the working channel or the distal extension of the working channel. The oblique channel preferably extends both distally and towards the working channel or the distal extension of the working channel. In particular, an angle of greater than 90° and smaller than 180°, preferably of greater than 100° and smaller than 170°, is formed between the supply channel and the oblique channel. Forming the clot dissecting channel as oblique channel, i.e. as inclined or angled channel, with respect to an axial direction of the working channel respectively of the insertion cord makes it possible to direct, guide and target the water jet across the suction path, which enables a suitable dissection of the clot into smaller clots. By arranging the oblique clot dissecting channel or an oblique portion of the clot dissecting channel only in, i.e. inside the distal tip, the clot dissecting channel can be suitably branched off from the fluid supply channel. The fluid supply channel preferably extends substantially parallel to the working channel. The oblique clot dissecting channel is thus preferably a rather short channel provided between the fluid supply channel and the working channel, which directs the water from the supply channel towards the working channel or the distal extension of the working channel.

According to an especially preferred embodiment, the clot dissecting channel is directed towards a distal opening of the working channel such that an ejecting direction of the water stream intersects a cross-sectional area defined by the distal opening of the working channel. By directing the water jet directly towards the cross-sectional area defined by the distal opening of the working channel, i.e. defined by the entrance of the working channel, it is possible to directly hit clots which block the working channel entrance. Therefore, the pointed and powerful fluid jet hits the clots directly and thus without being deflected, e.g. by the working channel wall surface. It is thus ensured that any clot, which has a size suitable for blocking the working channel, is directly hit, crushed and dissected, and can then be suctioned into the working channel without blocking the working channel entrance. Orienting the clot dissector channel directly towards the distal opening of the working channel may e.g. be realized by providing an oblique clot dissecting channel which has a small outlet or opening on a side of the working channel at or close to the distal opening of the working channel.

The clot dissecting channel may comprise a clot dissecting nozzle which is configured to generate the pointed and powerful fluid stream or jet that is able to dissect or crush clots or particles or debris. E.g. the clot dissecting nozzle may have a small outlet diameter of preferably less than 1 mm, more preferably less than 0.8 mm, especially preferred less than 0.6 mm, e.g. around 0.5 mm. In particular, when an irrigation pump is used which can provide fluid pressures around 5 bar, it is possible to generate the mentioned pointed and powerful fluid jet. The clot dissecting nozzle may decrease in diameter or may have a decreased diameter compared to a remainder of the clot dissecting channel, however may alternatively also have the same diameter as the remainder of the clot dissecting channel.

In a preferred embodiment, the endoscope further comprises a waterjet channel extending substantially parallel to the working channel and comprising a distal waterjet channel opening provided for distally ejecting a waterjet for rinsing the patient's body cavity. Related art endoscopes, in particular gastroscopes and colonoscopes, are often equipped with a waterjet channel and thus with a waterjet function. The waterjet function provides a forward pointing water jet that is used to clean mucosal walls inside the patient's body cavity or to clean bleeding to allow better visualization and inspection. The waterjet channel usually comprises a distal opening in the distal tip of the endoscope. Usually the waterjet channel and the working channel are oriented parallel to each other in the insertion cord and, in particular, in the distal tip.

According to an especially preferred embodiment, fluid is supplied to the clot dissecting channel and the waterjet channel through a common supply channel, preferably extending from a supply inlet provided at the handle, such that the clot dissecting channel and the waterjet channel branch off from the common supply channel in the distal tip. Hence, both of the channels, i.e. the waterjet channel and the clot dissecting channel may be supplied with fluid, e.g. water, by shared supply equipment. It is therefore possible to implement the clot dissecting channel according to the present disclosure in endoscopes with waterjet channels, in particular in gastroscopes and colonoscopes, by using the water supply equipment of the waterjet channel also for the clot dissecting channel. When the clot dissecting channel and the waterjet channel branch off from the common supply channel in the distal tip, only the distal tip has to be adapted to provide the clot dissecting channel. In other words, the clot dissecting channel according to the present disclosure can be provided on an endoscope which comprises also a waterjet function. However, the clot dissecting channel may be provided, according to the present disclosure, in an endoscope which does not presently have a waterjet channel, i.e. which does not have a waterjet function. Moreover, the clot dissecting channel may be provided, according to the present disclosure, as a standalone system running parallel to the waterjet system, i.e. to provide both the waterjet function and the clot dissecting function without sharing supply equipment or without sharing a fluids supply channel in the bending section of the endoscope.

In a further preferable design, a nozzle part in the form of a y-piece is inserted into a cavity formed in the distal tip, such that an entry portion of the nozzle part is in fluid communication with the common supply channel and the clot dissecting channel and the waterjet channel branch off from the common supply channel in the nozzle part. Providing a separated nozzle part, i.e. a standalone part which may be inserted into a cavity formed in the housing of the distal tip, especially has manufacturing advantages, in particular since it is a challenge to injection-mould a housing of the distal tip, which has an oblique clot dissecting channel. Therefore, from the manufacturing point of view it may be advantageous to provide the standalone nozzle part, in which the waterjet channel and the clot dissecting channel branch off from the common supply channel, and to mount said standalone part to the housing of the distal tip, in which the cavity for the nozzle part is provided.

A further preferred embodiment may be configured to comprise an activation means configured and provided for activating and deactivating the waterjet ejected through the clot dissecting channel. Advantageously, the clot dissecting functionality can thus be selectively activated and deactivated according to the present disclosure. According to an embodiment, the activation means may comprise a membrane covering an opening of the clot dissecting channel towards the working channel, the membrane being configured to open the opening and thus to activate the dissecting waterjet when the suction is applied in the working channel. According to another embodiment, the activation means may comprise a blocking device being movable between an inactivated state, in which the blocking device blocks water flow inside and through the clot dissecting channel, and an activated state, in which the blocking device releases water flow inside and through the clot dissecting channel. Especially preferred, in the activated state the blocking device may release fluid flow inside and through both the clot dissecting channel and the waterjet channel, in the inactivated state the blocking device may block fluid flow inside and through the clot dissecting channel, while releasing fluid flow inside and through the waterjet channel, and in a semi-activated state the blocking device releases fluid flow only inside and through the clot dissecting channel, while blocking fluid flow inside and through the waterjet channel.

In a particularly preferred design, the blocking device may be a latch connected to a flow control wire, which is configured to move the latch between the activated state, semi-activated state and the inactivated state, and preferably also intermediate positions between the activated state, the semi-activated state and the inactivated state. Thereby, the flow control wire may be guided from the handle to the distal tip via a wire pipe, which may be fixed in a channel extending throughout the insertion cord, thus forming a Bowden wire system. Furthermore, it may be advantageous, if a latch guide for guiding the latch may be formed in the distal tip of the endoscope, such that the latch is moved between the activated state, semi-activated state and the inactivated state by pushing and pulling the flow control wire. If the latch guide is L-shaped and the latch is made from a flexible material, it may be possible for the latch to bend about an angle in order to be movable within the latch guide. This may have the advantage that channels which are not parallel, e.g. the clot dissecting channel and the waterjet channel, can be selectively blocked with the one latch which is movable in the one latch guide. According to another alternative embodiment, as an activation means, a hinge door may be provided which can selectively prevent fluid flow through the waterjet channel or through the clot dissecting channel. The hinge door may be swiveled by an angle, e.g. by around 90°, in order to selectively enable or disable fluid flow through the waterjet channel and the clot dissecting channel.

According to a preferred embodiment, in case both the waterjet functionality and the clot dissecting functionality are provided and use the same common fluid supply channel, the activation means are configured such that the waterjet channel is closed when the clot dissecting channel is open. This enables a more powerful fluid jet being ejected from the clot dissecting channel. The valve may be positioned in or on the handle.

According to a further advantageous embodiment, the common supply channel may be divided into the clot dissecting channel and the waterjet channel by a junction structure, which may comprise a valve device, in particular a ball valve, for selectively releasing water flow into the clot dissection channel or into the waterjet channel.

As described hereinabove and with reference to the figures, the present disclosure relates to an additional feature in the form of a pointed and powerful fluid stream or jet guided and targeted across the suction path, which may be activated by a user if needed. Alternatively, the water jet provided by the clot dissecting channel may be configured to be always active when activating the waterjet functionality. The additional feature may be powered by ancillary equipment. In addition, it can be included in endoscopes with existing waterjet feature but can also be used as a standalone feature. The additional feature enables the user to potentially deconstruct particles to suction larger variety of particles, reduce the probability of the suction channel getting blocked or clogged as the particle size is reduced. Further, it gives a possibility to clean instruments before retracting. In particular, an inserted endoscopic tool can be cleaned using the clot dissecting functionality when it is retracted from the patient's body cavity. Loose material that is caught on the inserted endoscopic tool can thus be advantageously flushed away. Furthermore, the tip of an inserted tool can be lubricated using the clot dissecting functionality, for easier retraction of the inserted endoscopic tool. The lubricant may be water or another liquid. Furthermore, it is possible to clean a biopsy sample from the patient to confirm the correct probe was taken.

The present disclosure further relates to a system comprising the endoscope as described above and a video processing apparatus (VPA) couplable to the endoscope and capable of outputting a live image recorded by an image sensor of the endoscope on a display. The video processing apparatus may comprise a built-in display and/or a detachable display and/or may be couplable to an external display.

### BRIEF DESCRIPTION OF THE DRAWINGS

The disclosure will now be made in greater detail based on non-limiting examples and with reference to the schematic drawings on which:

FIG. 1 is a side view of an embodiment of a visualization system including an endoscope according to the present disclosure.

FIG. 2 is a perspective partial view of a distal tip of the endoscope of FIG. 1.

FIG. 3 is a schematic view of a fluid system of the endoscope of FIG. 1.

FIGS. 4 and 5 are partial cross-sectional views of the distal tip of the endoscope of FIG. 1 showing a clot dissecting arrangement in an activated and inactivated state, respectively.

FIG. 6 is a perspective view of an activation means of the clot dissecting arrangement of FIGS. 4 and 5.

FIGS. 7 to 9 are partial cross-sectional views of a variation of the distal tip of the endoscope of FIG. 1.

FIGS. 10 and 11 are partial cross-sectional view of another variation of the distal tip of the endoscope of FIG. 1 showing another clot dissecting arrangement in an inactivated and activated state, respectively.

FIG. 12 is a partial cross-sectional view of a further variation of the distal tip of the endoscope of FIG. 1 showing another clot dissecting arrangement.

FIGS. 13 and 14 are partial cross-sectional views of a still further variation of the distal tip of the endoscope of FIG. 1 showing another clot dissecting arrangement.

FIG. 15 is a schematic side view of another variation of the distal tip of the endoscope of FIG. 1 showing another clot dissecting arrangement.

FIG. 16 is a partial cross-sectional view a further variation of the distal tip of the endoscope of FIG. 1.

The figures are schematic in nature and serve only to understand the disclosure. Identical elements are marked with the same reference signs.

### DETAILED DESCRIPTION

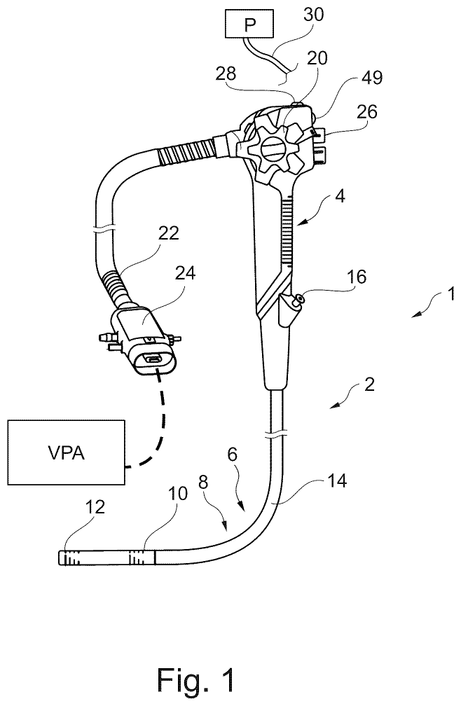

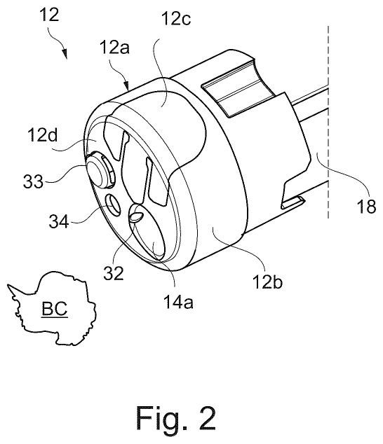

FIG. 1 shows a side view of a visualization system 1 including an endoscope 2 according to an embodiment of the present disclosure, which is preferably a single-use endoscope. The endoscope 2 comprises: a handle 4 and an insertion cord 6 extending distally from the handle 4. The insertion cord 6 is configured to be inserted into a patient's body cavity and comprises an insertion tube 8, a bending section 10 and a distal tip 12. The endoscope 2 further comprises a working channel 14, which extends from a biopsy port or working channel access port 16 provided at the handle 4 to the distal tip 12. The working channel 14 is comprised, in part, by a working channel tube 18 (shown in FIG. 2), which is arranged inside the handle 4, the insertion tube 8, and the bending section 10. The distal tip 12 comprises a housing that forms a portion of the working channel 14 and may be connected to the working channel tube 18. The handle 4 comprises a steering actuator 20, formed by two rotating units or handle wheels, for bending the bending section 10 of the insertion cord 6. Alternatively, the steering actuator 20 may comprise a lever. The endoscope 2 is connectable to a video processing apparatus (VPA), which is schematically illustrated via a connecting cable 22 comprising an electrical connector 24 insertable in a socket (not shown) of the VPA. Alternatively, the endoscope 2 may be wirelessly connectable to the VPA, for example by a wireless HDMI connection.

The handle's housing may comprise a plastic material. Furthermore, the housing preferably comprises two half-shells, a first half-shell and a second half-shell which are connectable with each other in a separation plane or dividing plane or split plane.

When a surgical instrument or tool is inserted into the working channel 14, the surgical instrument or tool is inserted into the working channel access port 16 and guided through a Y-connector (not shown) into the working channel tube 18.

The endoscope 2 further comprises a suction functionality, which can be activated by a suction button 26 formed on the handle 4 and configured to control a suction power through the working channel 14 of the endoscope 2. Further, the endoscope 2 comprises a vacuum port 28, which protrudes from or is embedded in the handle 4. The endoscope 2 is connectable to a suction pump P, which is schematically illustrated in FIG. 1, via a hose 30, which is connectable to the vacuum port 28 of the endoscope 2. Hence, medical staff can suction fluid, mucus, blood clots or other particles from the patient's body cavity during a surgical procedure by activating the suction pump P, such that the blood clots are extracted from the patient's body cavity through the working channel 14. In other words, the working channel 14 is used for enabling access into the patient's body cavity for surgical tools and as a suction channel for suctioning blood clots or the like out of the patient's body cavity. The size of blood clots BC which can be suctioned into the working channel 14 is determined by the size of a cross-section of the working channel 14, e.g. by a diameter of the working channel 14. If the size of a blood clot BC exceeds the size of the cross-section of the working channel 14 at least in portions, the blood clot BC might block the working channel 14, in particular a distal opening of the working channel 14. A clot dissection actuator 49 is also provided in the handle, as discussed below with reference to FIGS. 4 to 6.

To enhance the suction functionality, such that larger blood clots BC can be suctioned out of the patient's body cavity, the endoscope 2 according to the present disclosure is equipped with a blood clot dissecting functionality in the form of a clot dissecting channel 32 and a valve that enables or blocks fluid flow through the clot dissecting channel 32. The clot dissecting channel 32 is configured to eject a clot dissecting fluid stream. The clot dissecting fluid stream is a powerful waterjet or fluid stream configured to dissect blood clots. Thus, when the blood clot BC, the size of which exceeds the cross-section of the working channel 14, is suctioned into and blocks the working channel 14, the fluid stream ejected from the clot dissecting channel 32 splits the blood clot BC into smaller blood clots which can enter the working channel 14 due to their decreased size. Hence, the smaller blood clots can be suctioned out of the patient's body cavity.

FIG. 2 is a perspective view of the distal tip 12 illustrating a tip housing 12a comprising an opaque portion 12b and a translucent portion 12c. Inside the tip housing 12 are a camera and a light emitter. The camera receives light, and the light emitter transmits light, through windows formed by the translucent portion 12c. The distal tip 12 also comprises the clot dissecting channel 32, a cleaning nozzle 33, a waterjet channel 34, and a working channel distal opening 14a. A front wall 12d of the tip housing 12a comprises the working channel distal opening 14a, a distal opening of the waterjet channel 34 and the cleaning nozzle 33. An ejecting direction of the waterjet channel 34 may be substantially parallel to the working channel 14, in particular a center axis of the working channel 14. The waterjet channel 34 is used for rinsing the patient's body cavity, such that an operating site can be accessed and inspected properly. A proximal portion of the tip housing 12a is configured be attached to a distal segment of the bending section 10. Preferably, the bending section 10 comprises a single part including segments connected by living hinges, the single part being injection molded in one piece. The living hinges, or hinges, are thin portions of flexible polymer material that extend between pairs of segments.

As shown in FIG. 2, the clot dissecting channel 32 is directed and opens up towards the working channel 14, such that the clot dissecting fluid stream is targeted and guided across the working channel 14. In particular during use of the suction functionality, the clot dissecting channel 32 is configured to eject the clot dissecting fluid stream to split the blood clot BC before and while the blood clot BC enters the working channel 14.

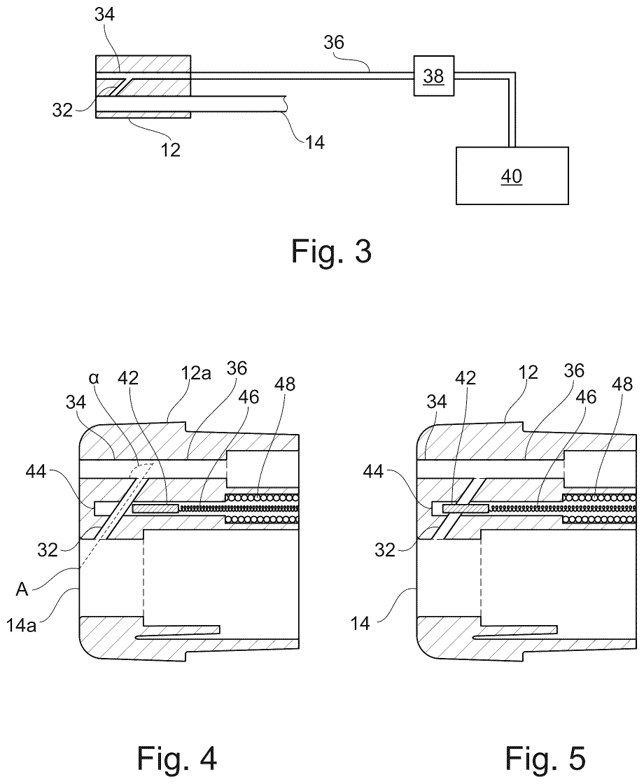

FIG. 3 is a schematic view of the components that provide the suction functionality. As shown, the clot dissecting channel 32 branches off from the waterjet channel 34 inside the distal tip 12, such that fluid flowing inside the waterjet channel 34 can flow into the clot dissecting channel 32 and discharged into the distal end of the working channel to dissect blood clots or other objects. The waterjet channel 34 and the clot dissecting channel 32 are supplied with fluid through a common supply channel 36, which is in fluid-communication with both the waterjet channel 34 and the clot dissecting channel 32. A proximal portion of the supply channel 36 is in fluid-communication with a supply inlet 38, which can be formed on the handle 4. Thus, medical staff can couple the supply channel 36 to a fluid supply or ancillary equipment 40, such as an irrigation pump or a syringe, via the supply inlet 38, such that fluid from the fluid supply equipment 40 enters the supply channel 36 via the supply inlet 38 and then flowing into the waterjet channel 34 and the clot dissecting channel 32. The fluid may consist of water or may comprised water and a gas or gases.

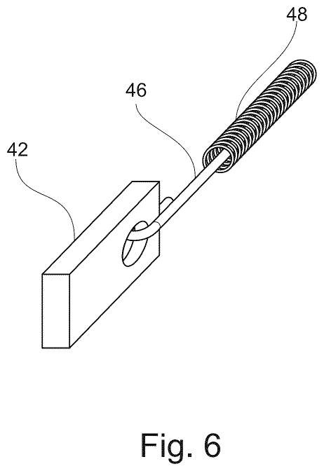

Referring now to FIGS. 4 and 5, a blocking device in the form of a latch 42 may be provided to prevent that the fluid is discharged via the clot dissecting channel 32 every time that the fluid is discharged from the waterjet channel 34. Thus, the latch 42 and the latch guide 44 form a valve. The latch 42 is guided in a latch guide 44 formed in the tip housing 12a and connected to a flow control wire 46 that translates inside a fixed wire pipe 48. As shown, the latch guide 44 is an elongate cavity that traverses the clot dissecting channel 32 so that when the latch 42 is translated distally it traverses the clot dissecting channel 32, as shown in FIG. 5, and thus blocks discharge of the clot dissecting fluid stream. A proximal end portion of the flow control wire 46 is connected to the clot dissection actuator 49, formed as a wheel or a lever, which is provided on the handle 4, such that medical staff can pull and push the flow control wire 46 to translate the latch 42 by actuating the clot dissection actuator 49. As shown in FIGS. 4 and 6, the flow control wire 46 extends within the fixed wire pipe 48 from the distal tip 12 to the clot dissection actuator 49, thus forming a Bowden wire system. The fixed wire pipe 48 may comprise a wire coil. As shown in FIGS. 4 and 5, the waterjet channel 34 has a distal end in the tip housing, which has a narrow distal portion and a wider proximal portion. The proximal portion is wider to facilitate insertion of a waterjet channel tube that provides a portion of the waterjet channel 34 that extends to the handle 4. Similarly, the portion of the working channel 14 formed by the tip housing has a proximal portion that is wider than the distal portion thereof and is configured to receive the working channel tube 18. Preferably, the inner diameter of the working channel tube is the same as the inner diameter of the distal portion of the working channel formed by the tip housing. The clot dissecting channel 32 comprises an axis A that is at an oblique angle inside the distal tip 12. The clot dissecting channel 32 extends both distally and towards the working channel 14, in particular towards an inner surface of the working channel 14. An angle a is formed between the supply channel 36 and the axis A of the clot dissecting channel 32. The angle a may be between 100° and 170°, preferably between 110° and 150°, and more preferably between 120° and 130°. The clot dissecting channel 32 is in particular directed towards the distal opening 14a of the working channel 14 such that an ejecting direction of the fluid stream (cf. axis A) intersects a cross-sectional area of the working channel at the distal opening 14a. Thereby, it is ensured that the fluid stream directly hits a blood clot BC stuck in the area of the distal opening 14a of the working channel 14, which enhances the clot dissecting functionality according to the present disclosure.

In FIG. 4, the latch 42 is in an activated state, where water flow inside the clot dissecting channel 32 is enabled and the clot dissecting fluid stream flows through the clot dissecting channel 32. When medical staff actuates the clot dissection actuator to stop discharging the clot dissecting fluid stream, the flow control wire 46 pushes the latch 42 in the distal direction to block fluid flow inside the clot dissecting channel 32 (cf. FIG. 5). Thus, the clot dissection actuator 49 translates the flow control wire 46 to move the latch 42 inside the latch guide 44 between an inactivated state, in which flow inside the clot dissecting channel 32 is blocked, and the activated state, in which flow inside the clot dissecting channel 32 is enabled.

FIG. 6 shows a perspective view of the latch 42. As shown, the latch 42, and thus the cross-section of the latch guide 44 in the tip housing, is rectangular. A rectangular cross-section may be preferred to minimize the space between the waterjet channel 34 and the working channel 14 and thereby enable a reduction in the size of the distal tip 12, which is desirable for many reasons, including navigation of the endoscope into narrow cavities. The latch 42 may, in one example, be provided with the large sides of the rectangle facing the waterjet channel 34 and the working channel 14. In another example, the latch 42 is cylindrical.

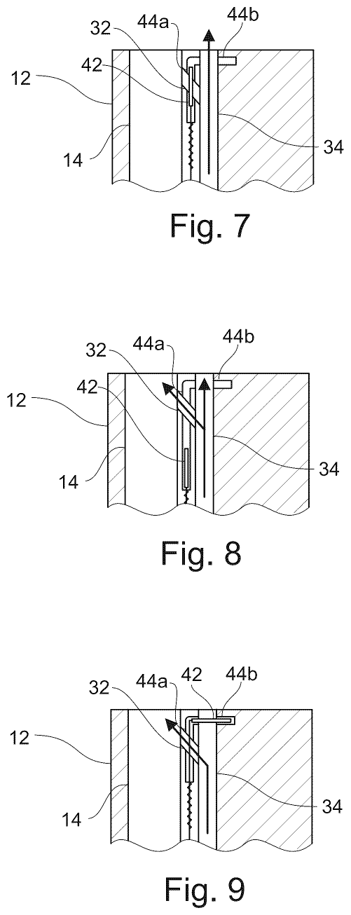

In the present embodiment the latch 42 is used as an activation means for activating and inactivating the clot dissecting functionality by enabling and blocking flow inside the clot dissecting channel 32. The clot dissecting functionality can thus be described as two-way functionality controlling flow through the clot dissecting channel 32, whereby flow in the waterjet channel 34 is affected when flow of fluid in the common supply channel 36 is diverted, or not, to the clot dissecting channel 32. FIGS. 7 to 9 show a variation of the present embodiment, in which the clot dissecting functionality can be described as three-way functionality. In the present variation, the latch 42 and the latch guide 44 are modified to enable flow only in the waterjet channel 34, only in the clot dissecting channel 32, and in both the waterjet channel 34 and the clot dissecting channel 32. The three-way functionality thus enables low and high fluid flow in the clot dissecting channel 32. When the fluid flows in both the waterjet channel 34 and the clot dissecting channel 32, flow in the clot dissecting channel 32 is low because some fluid flows in the waterjet channel 34. When flow in the waterjet channel 34 is blocked, flow in the dissecting channel 32 is high. Thus, the terms low and high are relative to the position of the latch 42. When flow is high, pressure is also higher because the cross-section of the clot dissecting channel 32 is not changed.

As can be seen in FIGS. 7 to 9, the latch guide 44 is formed in a L-shape having a first portion 44a extending in the proximal-distal direction and a second portion 44b extending perpendicularly to the first portion 44a. The first portion 44a is formed to cross the clot dissecting channel 32, whereas the second portion 44b is formed to cross the waterjet channel 34. The latch 42 is made from a flexible material such that the latch 42 can be moved within the whole latch guide 44, in particular the latch 42 can bend in order to be moved from the first portion 44a to the second portion 44b and vice versa. The flexible material may comprise silicone or other flexible polymer. The juncture of the first and second portions may be curved sufficiently to allow the flexible material to translate easily, without requiring too much force, between them. In FIG. 7 the latch 42 is arranged in the inactivated state, with the latch in the first portion 44a to block flow through the clot dissecting channel 32. However, flow through the waterjet channel 34 is enabled. In FIG. 8 the latch 42 is arranged in the activated state, with the latch positioned proximally of the clot dissecting channel 32. Thus, flow is enabled through the waterjet channel 34, and the clot dissecting channel 32 receives low flow. Due to the flexibility of the latch 42, it can be moved from the first portion 44a to the second portion 44b of the latch guide, as shown in FIG. 9. When the latch 42 is positioned in the second portion 44b, flow inside the waterjet channel 34 is blocked, while flow inside the clot dissecting channel 32 is enabled and receives all the fluid, thus high flow is enabled. In other words, the medical staff can select between the activated low flow state, the inactivated state and the activated high flow state. The medical staff can thus use all the fluid for irrigation, all the fluid for dissection, or can divide the fluid to perform irrigation and dissection simultaneously. The activated low flow state may be referred to as the activated state to reflect that flow in both the waterjet channel 34 and the clot dissecting channel 32 are enabled. By contrast, the activated high flow state may be referred to as the semi-activated state to reflect that flow in the waterjet channel 34 is blocked.

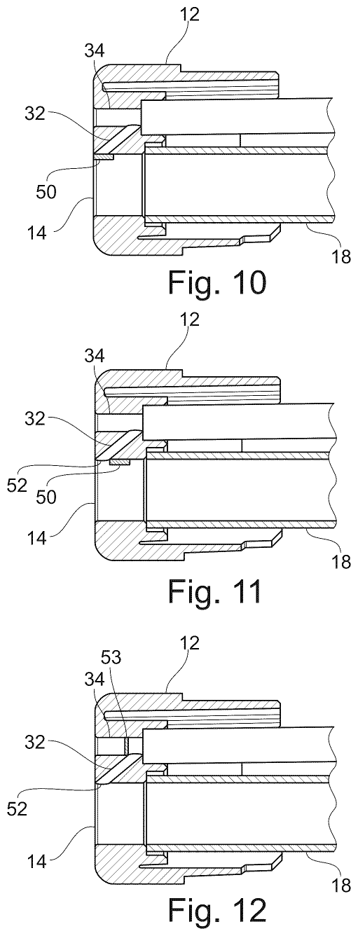

FIGS. 10 and 11 show another variation of the present embodiment of the endoscope 2. As discussed with reference to the endoscope, and variations thereof, described with reference to FIGS. 2 to 9, the endoscope 2 comprises the clot dissecting channel 32 fluidly coupled to the common supply channel 36 and activation means configured to activate or inactivate flow through the clot dissecting channel 32. In the present variation, as an activation means, a membrane 50 is arranged in the working channel 14. In the inactivated state shown in FIG. 10, the membrane 50 covers the distal opening 52 of the clot dissecting channel 32, such that no water is ejected from the clot dissecting channel 32. In the activated state shown in FIG. 11, the membrane 50 is retracted in the proximal direction, such that the distal opening 52 of the clot dissecting channel 32 is uncovered and flow is enabled through the clot dissecting channel 32.

The membrane 50 may be implemented as a x-fragm valve, which retracts or opens at a certain pressure. Hence, when the suction functionality of the working channel 14 is activated by operating the suction button 26, the pressure inside the working channel 14 decreases and the membrane 50 moves to the activated state. Thus, the membrane 50 blocks or enables flow in the clot dissecting channel 32 responsive to the pressure inside the working channel 14. When suction inside the working channel 14 is activated, the pressure drops and the membrane 50 opens the clot dissecting channel 32. The membrane 50 may comprise a slit or a pair of slits orthogonal to each other forming a cross. The slits' size, the material, and the thickness of the membrane are configured to create a gap between the edges of the slit/slits at a particular pressure created by fluid pressure on one side and vacuum on the other. The fluid pressure and vacuum are additive. Thus, the membrane automatically opens the slit/slits when there is fluid pressure and vacuum and closes the slit/slits when either or both sources of pressure are removed. The membrane 50 may be adhesively bonded onto the working channel or to an insert/nozzle inserted into the tip housing.

FIG. 12 shows the endoscope 2 according to a third embodiment. Thereby, as an activation means, a hinge door 53 is provided which can selectively prevent fluid flow through the waterjet channel 34 (as shown in FIG. 12) or through the clot dissecting channel 32. The hinge door 53 can be actuated via the clot dissection actuator 49 and can be swiveled in a clockwise direction by around 90° from the state shown in FIG. 12, in order to enable fluid flow through the waterjet channel 34 and to prevent fluid flow through the clot dissecting channel 32.

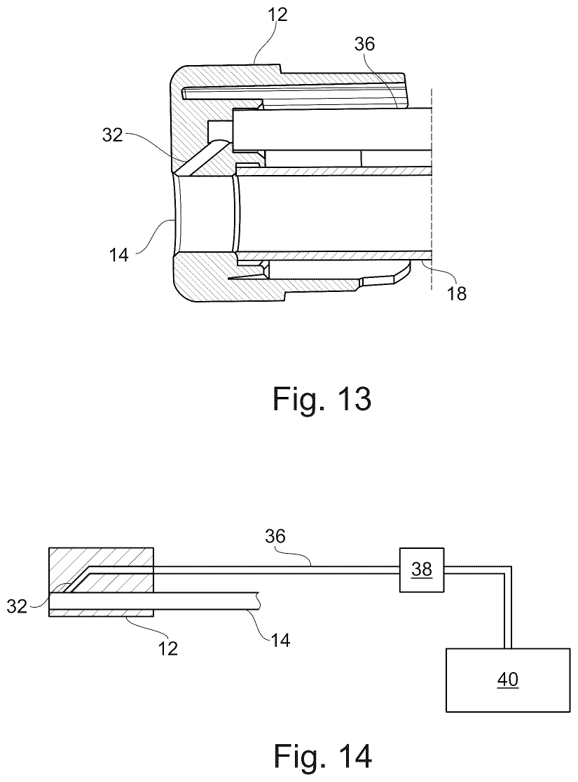

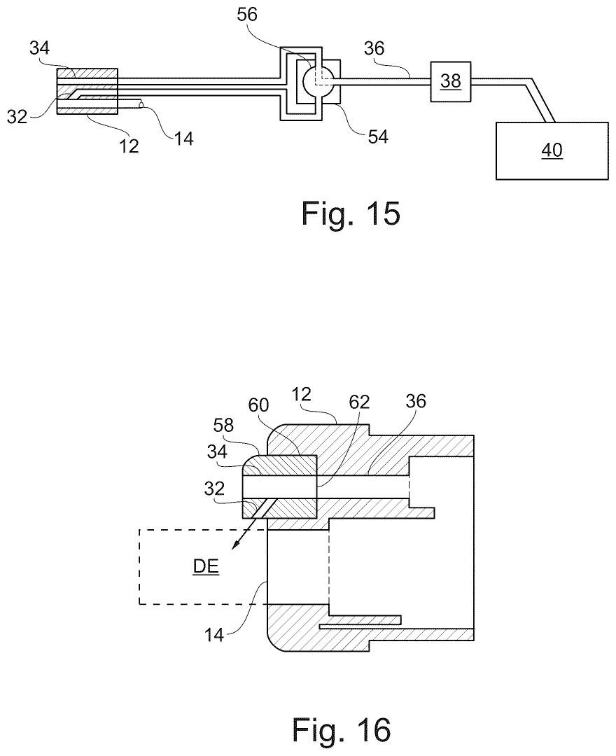

In the above described embodiment of the endoscope 2 and variations thereof, in addition to the clot dissecting channel 32, the waterjet channel 34 is formed in the distal tip 12. In a further variation, shown in FIGS. 13 and 14, the waterjet channel 34 is omitted and the common supply channel 36 supplies fluid only to the clot dissecting channel 32, as shown in FIGS. 13 and 14. A valve can be provided to block or enable flow through the clot dissecting channel 32. The valve can be located at the handle at or distally of the supply inlet 38. An example valve is shown in FIG. 15. The valve may be a quarter turn ball valve.

In a variation, shown in FIG. 15, the waterjet channel 34 is not omitted and the common supply channel 36 supplies fluid through a valve to clot dissecting channel 32 or the waterjet channel 34 or both. To this end, the clot dissecting channel 32 is fluidly coupled to a dedicated fluid supply channel that extends from its oblique portion proximally to the valve. The valve can be located at the handle at or distally of the supply inlet 38. The separate fluid supply channels may extend from the distal tip 12 to the handle 4, where a junction structure 54 comprising a valve mechanism 56, in particular a ball valve, is arranged. Upstream of the valve mechanism 54, the common supply channel 36 is connected via the supply inlet 38 to the supply equipment 40. Hence, fluid supplied by the supply equipment 40 enters the supply channel 36 via the supply inlet 38. At the junction structure 54, the supply channel 36 is split into the waterjet channel 34 and the clot dissecting channel 32. By operating the valve mechanism 54, fluid inside the supply channel 36 can be guided into the waterjet channel 34 and/or the clot dissecting channel 32. The valve can be configured to supply fluid to only the waterjet channel 34 or the clot dissecting channel 32.

In the above-described embodiment and variations thereof, the clot dissecting channel 32 and the optional waterjet channel 34 are formed integrally (in one-piece) with the housing 12a of the distal tip 12. Because the diameter of the tip housing is small, perhaps smaller than 4 mm and potentially smaller than 3 mm, molding an oblique channel can be challenging. In one example, the clot dissecting channel 32 may be formed by cutting the channel with a laser beam. This is facilitated by the clot dissecting fluid stream being directed to the opening of the working channel. The laser beam can thus be directed through the opening of the working channel toward the waterjet channel 34. The longitudinal channels 34, 44 can be molded directly in a mold comprising pins, whereby polymer is made to flow around the pins. Such molding can comprise the opaque and the translucent materials, which are injected in two steps but fused together to form the one-piece housing part. The clot dissecting channel 32 may also be formed by inserting a pin diagonally and traversing the channel forming pins. The diagonal pin has to be removed before the formed tip housing is removed from the channel forming pins. This process is not necessarily the most economical.

Alternatively, as can be seen in FIG. 16, the clot dissecting channel 32 and the waterjet channel 34 may be formed in a separate nozzle part 58, which is to be inserted into a respective cavity 60 provided in the distal tip 12, in particular in the housing 12a of the distal tip 12. Thereby, the nozzle part 58 is in the form of a y-piece. An inlet opening 62 of the nozzle part 58 is in fluid-communication with the supply channel 36, such that fluid flowing in the supply channel 36 can enter the nozzle part 58 via the inlet opening, before flowing into the waterjet channel 34 and the clot dissecting channel 32 formed in the nozzle part 58. The nozzle part 58 may be used in the variations shown in FIGS. 3 to 15 and may be particularly suitable for the provision of the valves in FIGS. 10 to 12 due to the channel openings being readily accessible to an assembler of the endoscope.

The clot dissecting channel 32 may, optionally, be directed and opens towards a distal extension DE of the working channel 14. The distal extension DE of the working channel 14 may be described as a virtual pipe-like structure, which is obtained when the working channel 14 is extended axially in the distal direction. In the embodiment of FIG. 16, the clot dissecting channel 32 is an oblique channel provided inside the distal tip 12, in particular in the nozzle part 58 being part of the distal tip 12, and guides the fluid stream towards the distal extension DE of the working channel 14. The clot dissecting channel 32 extends both distally and towards the distal extension of the working channel 14. Thus, the discharge opening of the clot dissecting channel 32 is provided distally of the front wall and aimed to the space distal of the working channel.

The following items are further variations and examples of the embodiments described with reference to the figures.

1. An endoscope (2) comprising: a handle (4) or interface; an insertion cord (6) extending from the handle (4) or interface, configured to be inserted into a patient's body cavity and comprising at least a distal tip (12); a working channel (14) which during a use of a suction is configured to let a fluid and a mucus and a clot suctioned from the patient's body cavity flow therethrough; and a clot dissecting channel (32) which is configured and provided to eject a fluid stream for dissecting the clot before or while the clot enters the working channel (14) during the use of the suction, the clot dissecting channel (32) being directed towards and opening up towards the working channel (14) or a distal extension (DE) of the working channel (14) in the distal tip (12).

2. The endoscope (2) according to item 1, wherein the clot dissecting channel (32) comprises or is an oblique channel inside the distal tip (12), the oblique channel guiding the fluid stream from a supply channel (36) towards the working channel (14) or the distal extension (DE) of the working channel (14).

3. The endoscope (2) according to item 2, wherein the supply channel (36) extends substantially parallel to the working channel (14).

4. The endoscope (2) according to item 2 or 3, wherein the oblique channel extends both distally and towards the working channel (14) or the distal extension (DE) of the working channel (14).

5. The endoscope (2) according to any one of items 2 to 4, wherein an angle of greater than 90° and smaller than 180°, preferably of greater than 100° and smaller than 170°, is formed between the supply channel (36) and the oblique channel.

6. The endoscope (2) according to any one of items 1 to 5, wherein the clot dissector channel (32) is directed towards a distal opening of the working channel (14) such that an ejecting direction of the fluid stream intersects a cross-sectional area defined by the distal opening of the working channel (14).

7. The endoscope (2) according to any one of the preceding items 1 to 6, further comprising a waterjet channel (34) extending substantially parallel to the working channel (14) and comprising a distal waterjet channel opening provided for distally ejecting a waterjet for rinsing the patient's body cavity.

8. The endoscope (2) according to item 7, wherein water is supplied to the clot dissecting channel (32) and the waterjet channel (34) through a common supply channel (36), such that the clot dissecting channel (32) and the waterjet channel (34) branch off from the common supply channel (36), preferably in the distal tip (12).

9. The endoscope (2) according to item 8, wherein a nozzle part (58) in the form of a y-piece is inserted into a cavity (60) formed in the distal tip (12), such that an entry portion (62) of the nozzle part (58) is in fluid communication with the common supply channel (36) and the clot dissecting channel (32) and the waterjet channel (34) branch off from the common supply channel (36) in the nozzle part (58).

10. The endoscope (2) according to any one of items 1 to 9, further comprising an activation means (42; 50; 56) configured and provided for activating and deactivating the waterjet ejected through the clot dissecting channel (32).

11. The endoscope (2) according to item 10, wherein the activation means comprises a membrane (50) covering an opening (52) of the clot dissecting channel (32) towards the working channel (14), the membrane (50) being configured to open the opening (52) and thus to activate the waterjet when the suction is applied in the working channel (14).

12. The endoscope (2) according to item 10, wherein the activation means comprises a blocking device (42) being movable between an inactivated state, in which the blocking device (42) blocks water flow inside and through the clot dissecting channel (32), and an activated state, in which the blocking device (42) releases water flow inside and through the clot dissecting channel (32).

13. The endoscope (2) according to item 12, wherein, in the activated state the blocking device (42) releases water flow inside and through both the clot dissecting channel (32) and the waterjet channel (34), in the inactivated state the blocking device (42) blocks water flow inside and through the clot dissecting channel (32), while releasing water flow inside and through the waterjet channel (34), and in a semi-activated state the blocking device (42) releases water flow only inside and through the clot dissecting channel (32), while blocking water flow inside and through the waterjet channel (34).

14. The endoscope (2) according to item 8, wherein the common supply channel (36) is divided into the clot dissecting channel (32) and the waterjet channel (34) by a junction structure (54), which comprises a valve device (56), in particular a ball valve, for selectively releasing water flow into the clot dissection channel (32) or into the waterjet channel (34).

15. System comprising an endoscope (2) according to any one of items 1 to 14 and a video processing apparatus (VPA).

### List of Reference Signs

2 endoscope

4 handle

6 insertion cord

8 insertion tube

10 bending section

12 distal tip

14 working channel

16 working channel access port

18 working channel tube

20 operating unit

22 connecting cable

24 electrical connector

26 suction button

28 vacuum port

30 hose

32 clot dissecting channel

34 waterjet channel

36 supply channel

38 supply inlet

40 supply equipment

42 latch

44 latch guide

44a first portion

44b second portion

46 flow control wire

48 wire pipe

49 clot dissection actuator

50 membrane

52 distal opening of clot dissecting channel

53 hinge door

54 junction structure

56 valve mechanism

58 nozzle part

60 cavity

62 inlet opening

VPA video processing apparatus

P pump

BC blood clot

DE distal extension

1. An endoscope comprising:
a handle or interface;
an insertion cord extending distally from the handle or interface and being configured to be inserted into a patient's body cavity, the insertion cord comprising a distal tip including a tip housing, the tip housing having a front wall;
a working channel extending from the handle to the tip housing, the working channel comprising a front opening at the front wall;
a fluid supply channel extending from the handle to the distal tip; and
a clot dissecting channel connected to the fluid supply channel inside the distal tip and having a distal opening configured to discharge at least a portion of a fluid supplied from the fluid supply channel towards the working channel or a distal extension of the working channel, the clot dissecting channel extending at an oblique angle to a longitudinal extent of the fluid supply channel.

2. The endoscope of claim 1, wherein the clot dissecting channel is directed towards the front opening of the working channel, a longitudinal axis of the clot dissecting channel intersecting the front opening of the working channel, wherein the endoscope further comprises:
a waterjet channel connected to the fluid supply channel in the distal tip, the waterjet channel comprising a distal waterjet channel opening, the waterjet channel being configured to discharge at least a portion of the fluid through the distal waterjet channel opening;
a latch guide traversing the clot dissecting channel;
a latch configured to translate in the latch guide; and
a flow control wire extending from the handle through the insertion cord and including a distal end connected to the latch, translation of the flow control wire translating the latch along the latch guide,
wherein when the latch is in a proximal position it does not traverse the clot dissecting channel and the fluid is enabled to flow therethrough, and
wherein when the latch traverses the clot dissecting channel the fluid is blocked from flowing therethrough.

3. The endoscope of claim 2, wherein the latch guide comprises a longitudinal portion traversing the clot dissecting channel and a transverse portion traversing the waterjet channel, wherein when the latch is in a distal position the latch traverses the waterjet channel and does not traverse the clot dissecting channel, thereby the fluid is blocked from flowing through the waterjet channel and is enabled to flow through the clot dissecting channel.

4. The endoscope of claim 1, wherein the fluid supply channel extends substantially parallel to the working channel.

5. The endoscope of claim 1, wherein the clot dissecting channel is directed towards the front opening of the working channel, a longitudinal axis of the clot dissecting channel intersecting the front opening of the working channel.

6. The endoscope of claim 1, wherein the oblique angle is greater than 100° and smaller than 170°.

7. The endoscope of claim 1, wherein the oblique angle is greater than 90° and smaller than 180°.

8. The endoscope of claim 1, wherein the oblique channel extends both distally and towards the working channel or the distal extension of the working channel.

9. The endoscope of claim 1, further comprising a waterjet channel connected to the fluid supply channel and extending substantially in parallel to the working channel, the waterjet channel comprising a distal waterjet channel opening, the waterjet channel being configured to discharge at least a portion of the fluid through the distal waterjet channel opening.

10. The endoscope of claim 9, wherein the waterjet channel and the clot dissecting channel are connected to the fluid supply channel in the distal tip.

11. The endoscope of claim 9, further comprising a valve positioned in or on the handle or interface, wherein the waterjet channel and the clot dissecting channel are connected to the fluid supply channel at the valve, and wherein the valve is configured to selectively enable fluid flow from the fluid supply channel into the clot dissection channel or into the waterjet channel.

12. The endoscope of claim 1, further comprising wherein a nozzle part in the form of a y-piece inserted into a cavity formed in the distal tip, the y-piece comprising an entry portion, a waterjet channel and the clot dissecting channel, the entry portion of the nozzle part being in fluid communication with the fluid supply channel, the clot dissecting channel and the waterjet channel branching off from the entry portion in the nozzle part, wherein the waterjet channel extends substantially in parallel to the working channel and comprises a distal waterjet channel opening, the waterjet channel being configured to discharge at least a portion of the fluid through the distal waterjet channel opening.

13. The endoscope of claim 1, further comprising an activation means configured to activate and deactivate discharge of the fluid through the clot dissecting channel.

14. The endoscope of claim 13, wherein the activation means comprises a blocking device being movable between an inactivated state, in which the blocking device blocks fluid flow inside and through the clot dissecting channel, and an activated state, in which the blocking device enables fluid flow inside and through the clot dissecting channel.

15. The endoscope of claim 14, wherein, in the activated state the blocking device enables fluid flow inside and through both the clot dissecting channel and the waterjet channel, wherein in the inactivated state the blocking device blocks fluid flow inside and through the clot dissecting channel while enabling fluid flow inside and through the waterjet channel, and in a semi-activated state the blocking device enables fluid flow only inside and through the clot dissecting channel while blocking fluid flow inside and through the waterjet channel.

16. The endoscope of claim 1, wherein the distal opening of the clot dissecting channel is adjacent a surface of the working channel, the endoscope further comprising a membrane covering the distal opening of the clot dissecting channel to block the distal opening of the clot dissecting channel when a suction is not applied in the working channel, the membrane allowing the fluid to flow through the distal opening of the clot dissecting channel when the suction is applied in the working channel.

17. A visualization system comprising the endoscope of claim 1 and a video processing apparatus.
